(19) United States
(12) Patent Application Publication
(10) Pub. No.: US 2026/0102171 A1
(43) Pub. Date: Apr. 16, 2026

(54) APPARATUS AND METHOD FOR IMPLEMENTATION OF BRIDGED STAPLE AND STERNAL PLATE

(71) Applicant: Arthrex, Inc., Naples, FL (US)

(72) Inventors: Stephan M. Bosch, Naples, FL (US); Corey Stewart, Naples, FL (US); Steven W. Rolenc, Naples, FL (US); Andrew C. Petry, Naples, FL (US)

(73) Assignee: Arthrex, Inc., Naples, FL (US)

(21) Appl. No.: 19/356,441

(22) Filed: Oct. 13, 2025

Related U.S. Application Data
(60) Provisional application No. 63/706,824, filed on Oct. 14, 2024.

(51) Int. Cl. A61B 17/17; A61B 17/00; A61B 17/064; A61B 17/80; A61B 90/00

(52) U.S. Cl. CPC A61B 17/1789; A61B 17/0642; A61B 17/8076; A61B 90/03; A61B 90/08; A61B 2017/00367; A61B 2017/00477; A61B 2017/0641; A61B 2090/034; A61B 2090/0811

(57) ABSTRACT

A surgical kit can include a drill plate assembly for drilling multiple pilot holes. The drill plate assembly can include a guide plate defining guide holes and a support plate defining an aperture. A gear assembly can include a receiving shaft extending through the aperture, a drive gear coupled with the receiving shaft, and follower gears engaged with the drive gear. Drill bits may be coupled with the follower gears. Rotation of the drive gear can cause rotation of the follower gears and rotation of the drill bits. The support plate may move toward the guide plate to move the drill bits through the guide holes to drill the multiple pilot holes. The kit can include an implant configured to be inserted into the multiple pilot holes. The implant may be a bridged, multi-prong staple and/or a sternal plate with fixation members.

### CROSS-REFERENCE TO RELATED APPLICATION

This application claims priority under 35 U.S.C. §119(e) and the benefit of U.S. Provisional Application No. 63/706,824 entitled APPARATUS AND METHOD FOR IMPLEMENTATION OF BRIDGED STAPLE, filed on Oct. 14, 2024, the entire disclosure of which is incorporated herein by reference.

### BACKGROUND

The present disclosure generally relates to an apparatus and a method for implementing a bridged staple and, more particularly, to an apparatus and method for concurrently drilling multiple pilot holes in bone for implementation of a multi-pronged, bridged staple. During surgical procedures, such as cardiovascular procedures, bone can be cut to access tissue for the surgical procedure. The bone is generally fixed together at the end of the surgical procedure with implants. In various implementations, the disclosure provides for devices and methods that may assist in closing incisions and reconnecting tissue to improve patient outcomes.

### SUMMARY

According to an aspect of the present disclosure, a surgical kit can include a drill plate assembly for concurrently or simultaneously drilling multiple pilot holes into bone. The drill plate assembly can include two plates, including a guide plate spaced from a support plate. The drill plate assembly may include a gear assembly between the two plates, including a drive gear operably coupled to the support plate and follower gears engaged with the drive gear 26. The drill plate assembly may also include multiple drill bits for drilling the multiple pilot holes. The drill plate assembly can include at least one biasing member between the plates. A force can be applied to the support plate to overcome a biasing force of the biasing member(s) to move the support plate toward the guide plate. Moving the plate together can move the drill bits through the guide plate and into the bone. A surgical drill can engage the drill plate assembly to drive the drive gear. Activation of the surgical drill can cause rotation of the drive gear, which can cause rotation of the follower gears of the gear assembly. The drill bits may be coupled with the follower gears, such that activation of the drill member causes rotation of the drill bits via the gear assembly.

The drill plate assembly can be part of a surgical kit with an implant that utilizes multiple pilot holes. In certain aspects, the implant can be a bridged staple. In additional or alternative aspects, the implant may be a sternal plate with fixation members. Each pilot hole for the implant may be drilled simultaneously by the drill plate assembly, which can assist with alignment of the pilot holes for the implant.

### BRIEF DESCRIPTION OF THE DRAWINGS

FIG. 1 is a schematic diagram of a surgical suite with a patient and a surgical kit including a drill device, a drill plate assembly, an inserter, and a bridged staple, according to the present disclosure;

FIG. 2 is a side perspective view of a drill plate assembly, according to the present disclosure;

FIG. 3 is a side elevational view of a drill plate assembly engaged with a drill member of a drill device, according to the present disclosure;

FIG. 4 is a side elevational view of a drill member engaged with a drill plate assembly with drill bits extending into bone in response to an applied force, according to the present disclosure;

FIG. 5 is a partial top plan view of a drill plate assembly with a support plate removed to illustrate a gear assembly, according to the present disclosure;

FIG. 6 is a cross-sectional view of a drive gear and a receiving shaft, where the receiving shaft has a receiving cavity with internal structures for engaging an end of a drill member of a drill device, according to the present disclosure;

FIG. 7 is a side elevational view of a drill bit coupled with a follower gear, according to the present disclosure;

FIG. 8 is a side perspective view of a drill member for a drill device, according to the present disclosure;

FIG. 9 is a side perspective view of a bridged staple, according to the present disclosure;

FIG. 10 is a front elevational view of a bridged staple in an initial, inwardly angled state, according to the present disclosure;

FIG. 11 is a rear elevational view of a bridged staple illustrated in a widened, insertion state in pilot holes in bones, according to the present disclosure;

FIG. 12 is a side perspective view of a bridged staple loaded on an inserter, according to the present disclosure;

FIG. 13 is a side elevational view of a drill member engaged with a drill plate assembly with drill bits extending through a sternal plate and into bone in response to an applied force, according to the present disclosure;

FIG. 14 is a partial top plan view of a drill plate assembly with a support plate removed to illustrate a gear assembly, according to the present disclosure;

FIG. 15 Is a schematic diagram of a sternum fixed together using a closure assembly, a bridged staple, and a sternal plate, according to the present disclosure;

FIG. 16 is a method of internally fixing bones together, according to the present disclosure;

FIG. 17 is a method of simultaneously drilling multiple pilot holes with a drill plate assembly, according to the present disclosure; and

FIG. 18 is a method of inserting a bridged staple, according to the present disclosure.

### DETAILED DESCRIPTION

In the following description, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

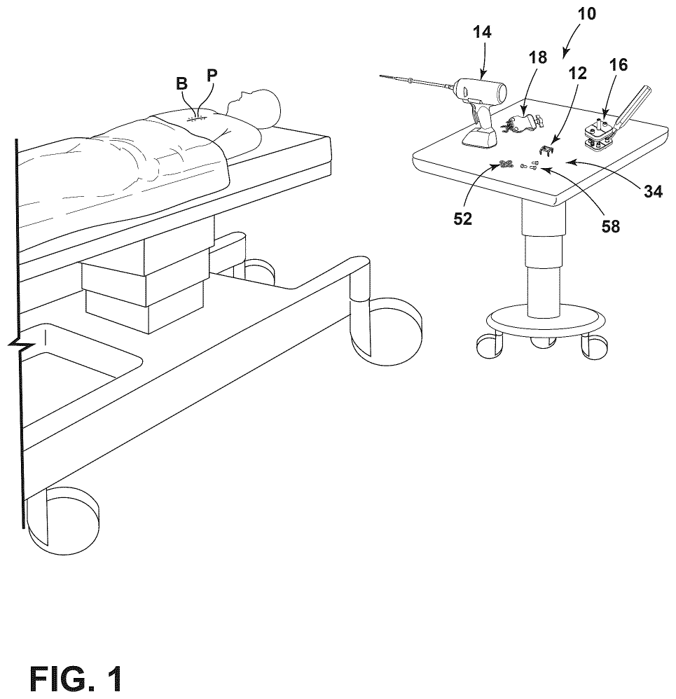

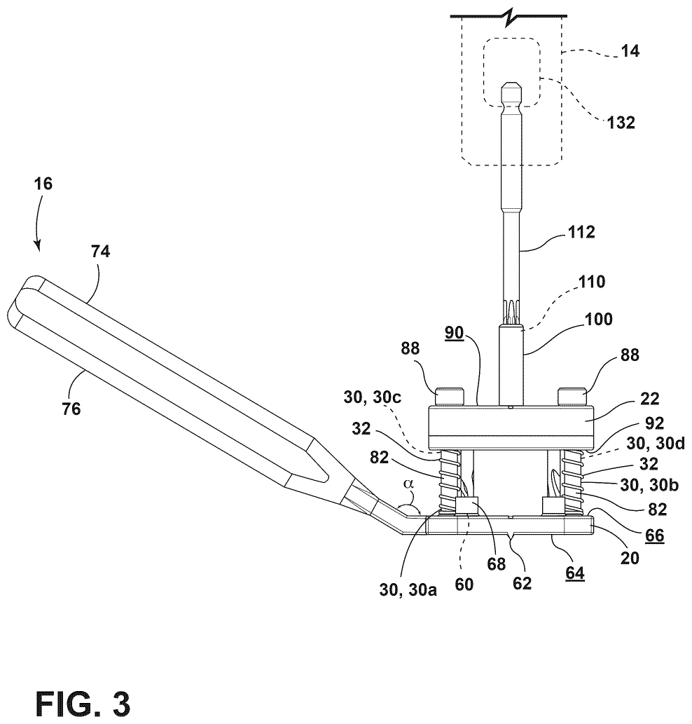

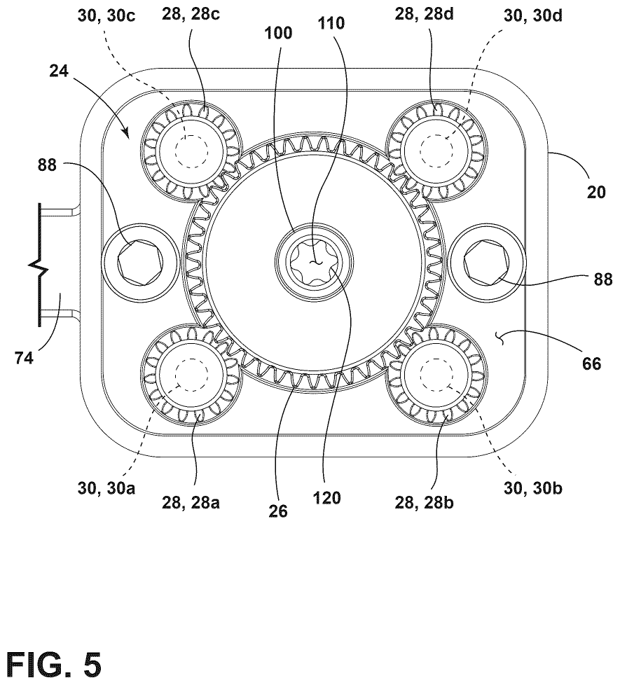

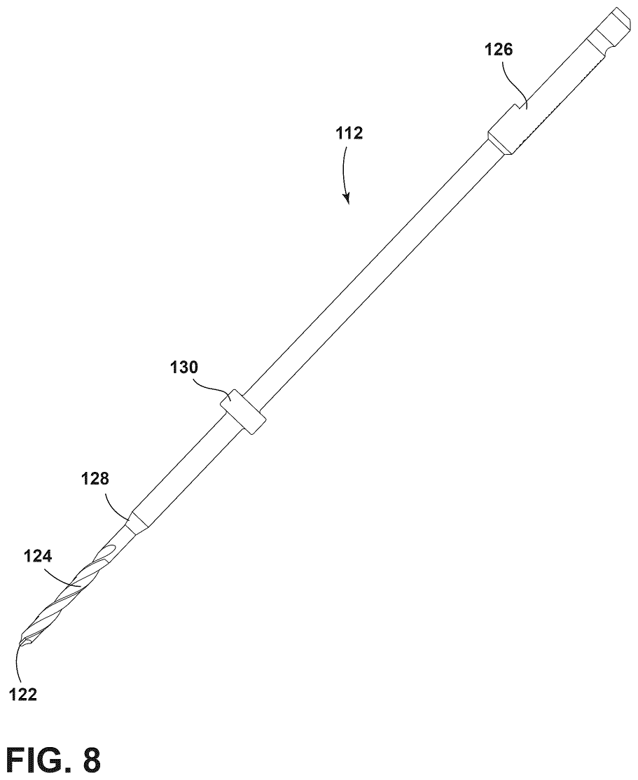

Referring to FIGS. 1-18, reference numeral 10 generally designates surgical kit 10, which can be utilized for forming or drilling pilot holes H in multiple bones B or bone portions B for multiple purposes or procedures, such as sternotomies and clamshell sternotomies. For example, the surgical kit 10 can be used for inserting a surgical staple 12 and internal fixing bones B together. The surgical kit 10 may include a drill device 14 or surgical drill 14, a drill plate assembly 16, an inserter 18, and the surgical, multi-pronged staple 12. The drill plate assembly 16 may be configured to concurrently drill multiple pilot holes H into the bones B. The drill plate assembly 16 can generally include two plates 20, 22, including a guide plate 20 spaced from a support plate 22. The drill plate assembly 16 may include a gear assembly 24 between the two plates 22, 24, including a drive gear 26 operably coupled to the support plate 22 and follower gears 28, including follower gears 28a-28d, engaged with the drive gear 26. The drill plate assembly 16 may also include multiple drill bits 30. The drill bits 30 can include drill bits 30a-30d (collectively referred to herein as drill bits 30), as illustrated in FIG. 5, or any number of drill bits 30 to correspond to the selected number of pilot holes H (e.g., n number of drill bits 30).

The drill device 14 may be configured to engage the drill plate assembly 16 to rotate the drive gear 26, which consequently rotates the follower gears 28 and the drill bits 30. The drill bits 30 may be configured to move into the bones B at a target area to drill the pilot holes H in response to a force F applied to the drill plate assembly 16. The drill plate assembly 16 may also include at least one biasing member 32 extending between the two plates 20, 22. The biasing member 32 may be configured to bias the support plate 22 away from the guide plate 20 (in a direction opposite the applied force F) and, consequently, draw the drill bits 30 away from or out of the target area.

The surgical kit 10 may be include a surgical implant 34. In certain aspects, the surgical implant 34 for the surgical kit 10 may include or be the surgical staple or bridged staple 12. The bridged staple 12 includes multiple prongs 40, which may include a first pair of prongs 40a, 40b and a second pair of prongs 40c, 40d. The first and second prongs 40a, 40b may be coupled via a first connector 42, and the third and fourth prongs 40c, 40d may be coupled via a second connector 44. At least one bridge 46 may couple the first and second connectors 42, 44 42, 44 to form the multi-pronged bridged staple 12. The bridged staple 12 may be biased toward a compressive state where the first pair of prongs 40a, 40b are angled inward, toward one another, and the second pair of prongs 40c, 40d are angled inward, toward one another forming two generally U-shaped staple portions 48, 50. The inserter 18 may be configured to adjust the prongs 40 of the bridged staple 12 outward to an opened or widened insertion state for inserting the bridged staple 12 into the multiple pilot holes H formed by the drill plate assembly 16. In response to the inserter 18 being disengaged from the bridged staple 12, the prongs 40 may adjust to or toward the compressive state to provide an inward, compressive force on the bones B.

The surgical implant 34 of the surgical kit 10 can also be surgical or sternal plates 52. For certain procedures or breaks in the bone B, at least one plate 52 may be utilized to fix the bone B or bone B portions together. Typically, plates 52 include a body 54 that can be positioned on an outer surface of the bone B. The body 54 defines apertures 56 for receiving fasteners, anchors, or fixation members 58 that may secure the plate 52 to the bone B. The body 54 may have a variety of configurations, and the apertures 56 may be arranged in one or more patterns on the body 54. The configuration of the plate 52, including shape of the body 54, number of apertures 56, arrangement of apertures 56, etc., may depend on the type of procedure, the bone B, etc.

The surgical kit 10 can include the surgical drill 14, the drill plate assembly 16, the surgical plate 52, and the fixation members 58. The drill plate assembly 16 may be configured to concurrently drill multiple pilot holes H into the bones B in an arrangement that matches or corresponds with the arrangement of the apertures 56. The drill plate assembly 16 may generally include the guide plate 20 spaced from the support plate 22 with the gear assembly 24 between the two plates 22, 24. The drill plate assembly 16 may also include multiple drill bits 30 supported by at least one of the plates 22, 24. The drill bits 30 may be arranged in any pattern to match the apertures 56 in the plates 52, and the gear assembly 24 may include any practicable number of gears 26, 28 to cause the drill bits 30 to rotate. Accordingly, the drill plate assembly 16 may include at least two drill bits 30 but may include any number of drill bits 30 without departing from the teachings herein.

The drill plate assembly 16 is configured to drill multiple pilot holes H concurrently that align with the pattern, arrangement, or spacing of the apertures 56 of the plate 52. The drill bits 30 may be positioned in the apertures 56 during the drilling process or the plate 52 may be positioned on the surgical site after the pilot holes H have been drilled. The plate 52 may be positioned at the surgical site with the apertures 56 aligned with the pilot holes H. The fixation members 58 may then be inserted through the apertures 56 and into the bone B to fix the plate 52 to the bone B. The fixation members 58 may be screws, pins, staples, the bridged staple 12, or other similar device without departing from the teachings herein.

As shown, the surgical kit 10 may generally be configured for concurrently drilling multiple pilot holes H in adjacent bone portions B to insert the multi-pronged bridged staple 12 or implant the plate 52 and provide compressive fixation of the adjacent bone portions B. In non-limiting examples, bones B or bone portions B may be cut or broken for access to tissue. For example, the bones B may be the sternum or a rib bone for accessing tissue, such as the heart for various surgical procedures. Upon completion of the procedure to the tissue, the bridged staple 12 or the plate 52 may be used to internally fix the bones B together and apply the compressive force. In certain aspects, the bridged staple 12 and the plate 52 may be considered an implant 34 for providing internal fixation of two bones B or bone portions B to provide continuous fixation and compression between the two bones B. The surgical kit 10 may be used in cardiovascular procedures, as well as other procedures where the bridged staple 12 or the plate 52 may be advantageous for fixing bones B together.

As demonstrated in various examples, the drill plate assembly 16 may provide for the location and application of multiple drill bits 30. Without the benefit of the disclosed apparatus 16, the individual holes associated with the prongs of a staple or openings in a plate would be placed and cut individually. Even with the benefit of a guide or template to cut the holes utilized to seat two or more prongs of the staples or provide two or more openings for plates, the surgical drill is moved between each of the successive holes of the drill guide to sequentially drill the pilot holes H into the bones B or tissue. This process can be both time-consuming and cause variations among the positions of each pilot hole H due to changes in the position of the guide. Such changes may be the result of the guide shifting due to contact by a tool or user. Similarly, the repeated engagement of a drill bit with a guide may result in inadvertent binding or snagging between the drill bit and the guide openings. Such issues may result in reduced accuracy and increased procedure times.

The surgical kit 10 provided herein may provide for the concurrent actuation and drilling of multiple pilot holes H simultaneously in a predefined arrangement for the insertion of the multi-pronged bridged staple 12 or implanting of the surgical plate 52. The surgical kit 10 may be advantageous for increasing the efficiency of the surgical process by drilling the pilot holes H for all prongs 40 in a single bridged staple 12 and/or all apertures 56 of the surgical plate 52 simultaneously. Further, the use of the bridged staple 12 or plate 52 may be advantageous for using fewer staples in the surgical process, as well as for providing constant compression between the bones B with reduced translational forces.

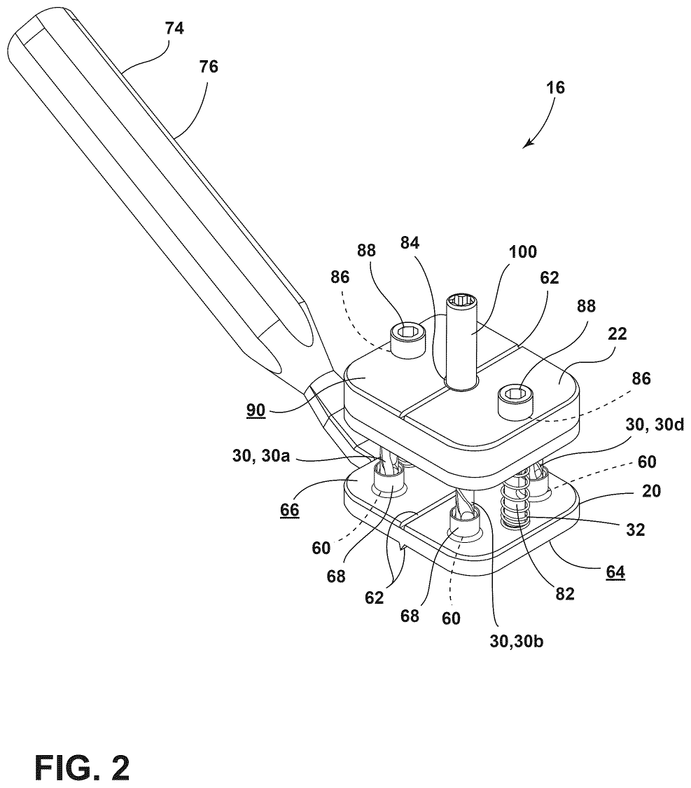

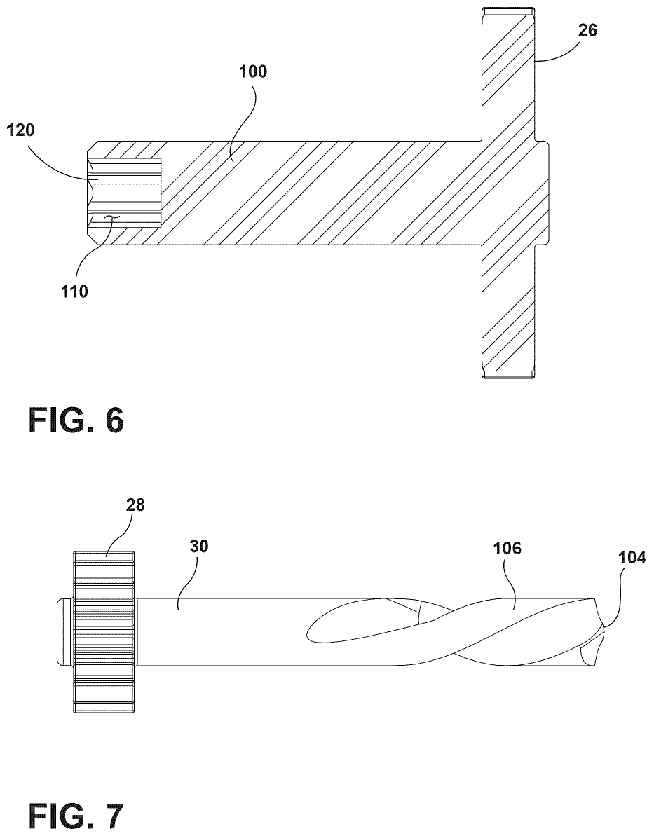

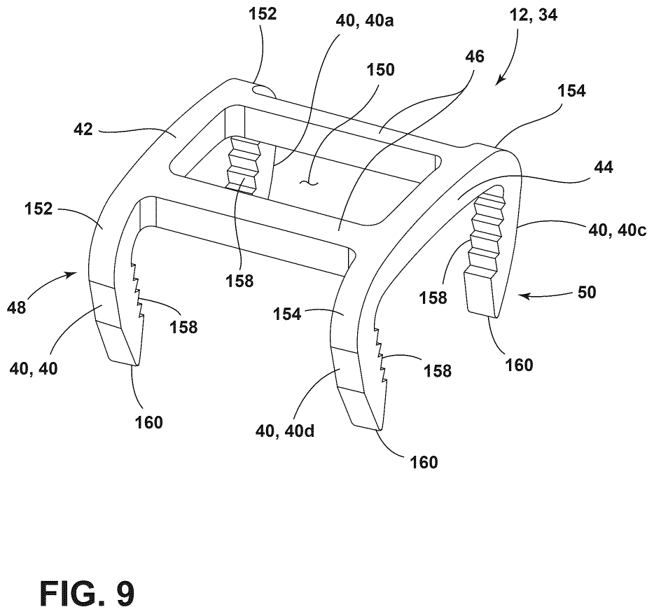

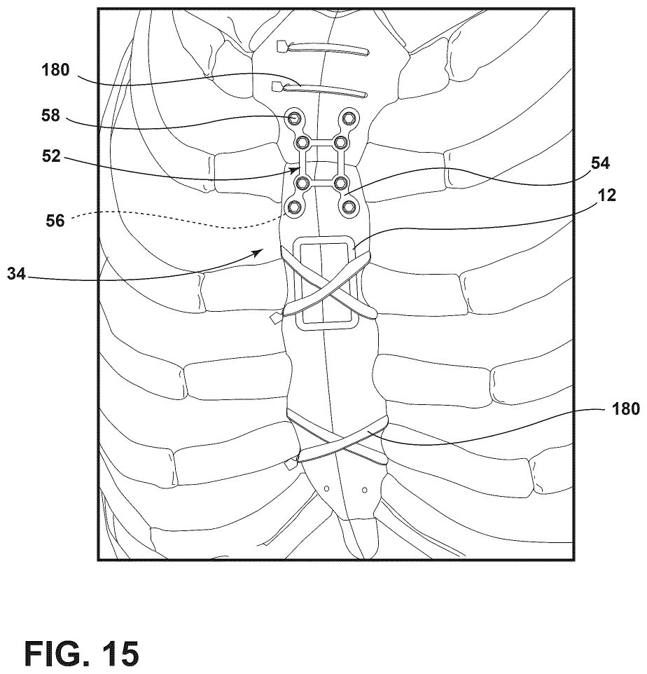

Referring to FIGS. 2-4, the drill plate assembly 16 may include the guide plate 20, which is configured to be positioned against the target area, such as the bones B to be drilled. The guide plate 20 may define guide holes 60 through which the drill bits 30 are configured to extend, respectively. In the illustrated configuration, the guide plate 20 has a quadrilateral shape, and the guide holes 60 are arranged proximate to corners of the guide plate 20. This arrangement may correspond with the prongs 40 of the bridged staple 12 (FIG. 9) or the apertures 56 of the plate 52 (FIG. 15), as well as for positioning the drill bits 30 about the drive gear 26 (FIGS. 5 and 6) as described herein. However, other arrangements are contemplated, including linear arrangements, parallel arrangements, diagonal arrangements, offset or staggered arrangements, box patterns, “H” patterns, or combinations thereof. The guide plate 20 may include at least one alignment indicator 62. The alignment indicator 62 may be configured to align with the cut between the bones B to align the guide holes 60 and the drill bits 30 for forming the pilot holes H. The indicator 62 may also be advantageous for positioning the pilot holes H generally equidistant from the cut.

The guide plate 20 includes an engagement surface 64, which is illustrated as a lower surface, and which may be configured to abut or engage the target area. In certain aspects, the engagement surface 64 of the guide plate 20 may be generally flat or planar. The flat configuration may be advantageous for positioning the drill bits 30 at a predefined distance from the bones B. It is also contemplated that the engagement surface 64 and/or the guide plate 20 may have different shapes to correspond to particular bony locations. For example, if the drill plate assembly 16 is to be used on a rib bone, the engagement surface 64 of the guide plate 20 may define a slight curvature for receiving the rib bone and assisting with maintaining the drill plate assembly 16 in the selected area. The guide plate 20 may have rounded edges and corners, which may assist with reducing friction or “catching” on surrounding tissue as the drill plate assembly 16 is moved and positioned against the target area.

The guide plate 20 may define multiple guide holes 60, such as the four guide holes 60 in the illustrated configuration. Typically, the guide plate 20 defines a number of guide holes 60 which corresponds with the number of drill bits 30 and the number of prongs 40 in the bridged staple 12 (FIG. 9) or aperture 56 in the sternal plate 52 (FIG. 15). The guide holes 60 extend through the guide plate 20 from an inner surface 66, facing the support plate 22, to the engagement surface 64. The drill bits 30 are configured to extend and move in a first direction (e.g., an insertion direction) through the guide holes 60 to form the pilot holes H and in a second direction (e.g., a removal direction) to be removed from the target area as described herein.

The drill plate assembly 16 may also include receivers 68 extending from the inner surface 66 of the guide plate 20 and toward the support plate 22. The receivers 68 may extend around the guide holes 60, respectively, and form channels for receiving and guiding the drill bits 30 through the guide holes 60. The receivers 68 generally extend along a perimeter of the guide holes 60 with the channels forming an extension of the guide holes 60. The receivers 68 may extend from the inner surface 66 and operate as a stopper for movement of the support plate 22 as described herein.

Referring still to FIG. 2-4, the drill plate assembly 16 may include a handle 74, which may extend from the guide plate 20. Generally, the handle 74 may extend at an obtuse angle α relative to the inner surface 66 of the guide plate 20. This angled orientation may provide an ergonomic grasping location for the user (e.g., the surgeon or member of the surgical team) for moving the drill plate assembly 16 to the target area and holding the drill plate assembly 16 during a drilling process. The angled orientation of the handle 74 may also assist the user with holding the drill plate assembly 16 in one hand and the drill device 14 in a second hand to drill the pilot holes H.

The handle 74 may be generally cylindrical with grooves 76 or ridges extending along a longitudinal extent thereof. The grooves 76 or ridges may provide a texture for assisting the user with maintaining a grip on the handle 74 while wearing gloves. Further, the handle 74 may include one groove 76 or ridge on each side, which may provide tactile feedback for the user related to the orientation of the drill plate assembly 16.

Referring still to FIGS. 2-4, the drill plate assembly 16 may also include the support plate 22, which may generally be spaced above the guide plate 20 and farther from the target area. The support plate 22 may be operably coupled to the guide plate 20 by the drill bits 30 and at least one support shaft 82. The support plate 22 may have an increased thickness compared to the guide plate 20 for supporting various components of the drill plate assembly 16. In various aspects, the support plate 22 has a quadrilateral shape that corresponds with the shape of the guide plate 20. The corresponding shapes may be advantageous for aligning components operably coupled or disposed on the support plate 22 with components or features of the guide plate 20 while minimizing an overall footprint of the drill plate assembly 16. The support plate 22 may be configured to move relative to the guide plate 20 (e.g., toward and away) and also transfer an applied force F from the drill device 14 to the drill bits 30.

The support plate 22 may define a receiving aperture 84 and through holes 86. In the illustrated configuration, the through holes 86 and the receiving aperture 84 are defined in a generally linear arrangement and aligned with the longitudinal extent of the handle 74. In the example shown, the receiving aperture 84 is arranged on the alignment indicator 62 on the support plate 22 with the through holes 86 on either side of the alignment indicator 62. However, other configurations are contemplated without departing from the teachings herein. For example, the through holes 86 may be arranged in a linear configuration with the receiving aperture 84 in a direction normal to the longitudinal extent of the handle 74. It may be advantageous for the through holes 86 to be arranged on opposing sides or ends of the support plate 22 to receive the support shafts 82 for guiding movement of the support plate 22.

The drill plate assembly 16 may include the support shafts 82, extending between the guide plate 20 and the support plate 22. In certain aspects, the support shafts 82 may be fixedly coupled with the guide plate 20 and extend through the through holes 86 of the support plate 22, allowing the support plate 22 to move relative to the support shafts 82. Caps 88 may be coupled to the support shafts 82 proximate to an outer surface 90 of the support plate 22 to maintain the engagement between the support shafts 82 and the support plate 22. The caps 88 may also provide an outer limit for movement of the support plate 22 away from the guide plate 20. The support shafts 82 may be arranged on opposing sides or ends of the support plate 22 to facilitate more even movement of the support plate 22 along the support shafts 82.

The drill plate assembly 16 may include at least one biasing member 32 extending between the guide plate 20 and the support plate 22. Generally, the drill plate assembly 16 may include at least two biasing members 32. As in the illustrated configuration, the biasing members 32 can be configured as coil springs extending around the support shafts 82, respectively. The biasing members 32 are configured to bias the support plate 22 away from the guide plate 20. Accordingly, the support plate 22 may be configured to move against the biasing force of the biasing members 32 and toward the guide plate 20, and the biasing member 32 may assist in returning the support plate 22 to an original state or position. The two biasing members 32 arranged at the opposing sides or ends of the support plate 22 may assist in providing a more even biasing force on the support plate 22 for guiding movement of the support plate 22 and distributing the applied force F across the drill plate assembly 16.

The support plate 22 may be configured to move relative to the guide plate 20 in response to the force F applied to the support plate 22. In certain aspects, the drill device 14 (FIG. 1) may be configured to engage the drill plate assembly 16 proximate to the outer surface 90 of the support plate 22. The drill device 14 may be utilized to apply the force F to the support plate 22, which can overcome the biasing force of the biasing members 32 to move the support plate 22 along the support shafts 82. The support plate 22 may be configured to move toward the guide plate 20. In certain aspects, the receivers 68 may operate as stoppers for defining a limit to the movement of the support plate 22. Accordingly, the support plate 22 may be configured to move toward the guide plate 20 and abut the receivers 68, maintaining a space between the support plate 22 and the guide plate 20 for components therebetween, such as the gear assembly 24. The height of the receivers 68 may be adjusted or may differ to change a depth of insertion of the drill bits 30.

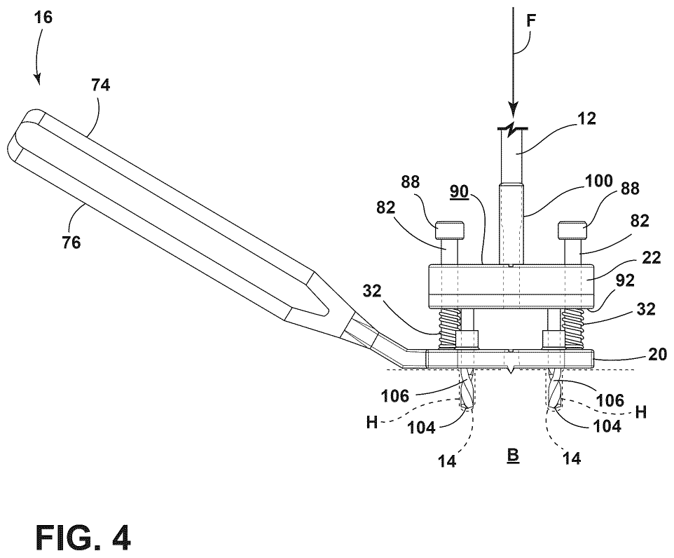

Referring now to FIGS. 4-7, the drill plate assembly 16 may include the gear assembly 24 or gear set 24 for transferring rotational forces through multiple components and, ultimately, forming the pilot holes H. The gear assembly 24 may be positioned substantially between the support plate 22 and the guide plate 20. The gear assembly 24 may be operably coupled with an inner surface 92 of the support plate 22, such that movement of the support plate 22 toward the guide plate 20 causes movement of the gear assembly 24 toward the guide plate 20. In this way, the gear assembly 24 may be configured to move with the support plate 22 and rotate separately from the support plate 22.

The gear assembly 24 may include the drive gear 26 and multiple follower gears 28 engaged with the drive gear 26. The drill plate assembly 16 may include the same number of follower gears 28 as drill bits 30 (e.g., n number of follower gears). The drive gear 26 may be positioned proximate to the inner surface 92 of the support plate 22 and include or be coupled with a receiving shaft 100 that is configured to extend through the receiving aperture 84 of the support plate 22. In this regard, the gear assembly 24 may be configured to extend through the support plate 22 and away from an upper or outer surface 90 of the support plate 22.

Referring to FIGS. 4-6, the drive gear 26 may be the largest gear in the gear assembly 24 for engaging each of the follower gears 28. As shown, the drive gear 26 may be centrally located between the follower gears 28 to engage each of the follower gears 28 simultaneously for driving the drill bits 30 simultaneously. The drive gear 26 may have a variety of sizes or configurations depending on the overall configuration of the drill plate assembly 16. In the illustrated example, the drive gear 26 may have an outer diameter of between about 0.5 inches and about one inch with between about 35 and about 50 teeth. A root diameter and a pitch diameter may each be between about 0.5 inches and 0.75 inches, with the root diameter being smaller than the pitch diameter. The drive gear 26 (e.g., a gear portion of the drive gear 26 separate from the receiving shaft 100) may have a thickness of less than about 0.25 inches. With the receiving shaft 100, the drive gear 26 may have a length between about 0.5 inches and one inch. The gear portion may have a thickness that is less than a height of the receivers 68 for maintaining the space between the drive gear 26 and the inner surface 66 of the guide plate 20 when the support plate 22 is moved to abut the receivers 68 (FIG. 4) as described herein.

As shown, the drive gear 26 may engage multiple follower gears 28. In the illustrated configuration of FIGS. 4 and 5, the drill plate assembly 16 may include four follower gears 28a-28d corresponding to the four drill bits 30a-30d in the drill plate assembly 16. Depending on the configuration of the gear assembly 24, the drive gear 26 may be off-center relative to the drill guide assembly 16 and/or the follower gears 28. The follower gears 28 may be arranged at corners of the support plate 22, but the arrangement may be based on the pattern for the pilot holes H and the bridged staple 12 (FIG. 9) or the sternal plate 52 (FIG. 15). The follower gears 28 may be arranged at intervals or equal spacing around the drive gear 26, which may provide for consistent and simultaneous rotation of the follower gears 28. Though four follow gears 28 are demonstrated and shown, different configurations may be implemented with one, two, three, five, or more follower gears depending on the procedure and number of pilot holes H. Each follower gear 28 may directly engage and be driven by the drive gear 26. Alternatively, at least one follower gear 28 may be indirectly driven by the drive gear 26, as described further herein.

Referring to FIGS. 4, 5, and 7, each of the follower gears 28 may be the same size to provide the same rotation to the drill bits 30. In non-limiting examples, the follower gears 28 may have an outer diameter between about 0.1 inches and about 0.5 inches with between about 10 and 20 teeth. A root diameter and a pitch diameter may each be less than 0.25 inches, with the root diameter being smaller than the pitch diameter. The follower gears 28 may have a thickness of less than 0.25 inches and may have a same thickness as the gear portion of the drive gear 26 for maintaining separation from the guide plate 20 when the support plate 22 is moved to abut the receivers 68. The inner surface 92 of the support plate 22 may be generally planar or flat and the gear assembly 24 may be coupled to the inner surface 92. Additionally or alternatively, the inner surface 92 may define one or more recessed regions with the gear assembly 24 operably coupled to the inner surface 92 in the recessed region(s) for increasing space between the gear assembly 24 and the guide plate 20. This may be advantageous for reducing any significant impact on the rotation of the gear assembly 24 in response to the movement or positioning of the support plate 22.

The drill bits 30 may extend from the follower gears 28. The drill bits 30 may also be referred to as follower drill bits 30 or secondary drill bits 30. Each drill bit 30 may be operably coupled with and extend from a respective follower gear 28. Accordingly, the drill bits 30 may be arranged around the follower gear 26, such as proximate to the corners of the guide plate 20 to extend through the receivers 68 and the guide holes 60. The drill bits 30 may have a length to form pilot holes H of a sufficient or selected depth in the bones B. Generally, the drill bits 30 may have a length of between about 0.5 inches and about one inch, such as about 0.775 inches. Each drill bit 30 may have a thickness or width that is less than the outer diameter of the corresponding follower gear 28 and may be also less than the root diameter of the corresponding follower gear 28.

Each drill bit 30 may include or define a point 104 and flutes 106 with the flutes 106 extending along at least a portion of a length of the drill bit 30 to or toward the follower gear 28. In non-limiting examples, the flutes 106 may have a length of less than about 0.75 inches, such as about 0.49 inches, and may have a helix of about 27°. The size and arrangement of the gears 26, 28 and the configuration of the drill bits 30 may depend on the bone B or location for drilling the pilot holes H.

Referring now to FIGS. 4-6 and 8, the gear assembly 24 may be configured to be engaged by the drill device 14 for causing rotation of the gears 26, 28 and the drill bits 30. The receiving shaft 100 may define a receiving cavity 110 for receiving a drill member 112 of the drill device 14. The drill member 112 may also be referred to as a driving drill bit or primary drill bit. The receiving cavity 110 may define ridges, grooves, or other engagement structures or features 120 along an interior surface thereof for mating with an end of the drill member 112. For example, the end of the drill member 112 may define or include a point 122 and flutes 124. The interior surface forming the receiving cavity 110 may define a shape for receiving the point 122 and the flutes 124 and forming a mating or interlocking engagement between the receiving shaft 100 and the drill member 112. This mating or interlocking engagement may assist with transferring rotation from the drill member 112 to the receiving shaft 100. Moreover, this engagement may maximize efficiency in transferring the rotational force from the drill member 112 by reducing the rotation of the drill member 112 separate from the receiving shaft 100 (e.g., rotation of the drill member 112 within the receiving cavity 110 that does not rotate the receiving shaft 100).

The drill member 112 of the drill device 14 may have a variety of configurations, so long as the end of the drill member 112 mates or interlocks with the receiving cavity 110. In the illustrated configuration, the drill member 112 has a length sufficient to engage the drill device 14 and the drill plate assembly 16, which may be between about four inches and about five inches. The drill member 112 may include a proximal feature 126 for engaging the drill device 14 at one end and the point 122 at the opposing end for engaging the drill plate assembly 16. The point 122 may include multiple facets with at least a primary relief and a secondary relief. The different facets and/or reliefs may be used to mate or interlock with the drill plate assembly 16 for driving the drill bits 30.

The drill member 112 may include the flute 124 extending from the point 122 and along the length of the drill member 112. The flute 124 may extend less than one inch in length, such as about 0.59 inches, and may have a helix of about 23°. The point 122 may define an angle, such as an acute angle, which may be about 75°. The drill member 112 may define a shoulder 128, where the drill member 112 widens. A distance from the point 104 to the shoulder may be less than about one inch, such as about 0.86 inches. Additionally, in non-limiting examples, the drill member 112 may include a stopper 130. The stopper 130 may be coupled with or integrally formed with the drill member 112. The drill member 112 may be configured to be utilized with the drill plate assembly 16 for indirectly forming the pilot holes H and may also be used for directly forming the pilot holes H.

Referring to FIGS. 1-8, in operation, the drill device 14 and the drill plate assembly 16 may be utilized to simultaneously or concurrently drill multiple (e.g., two or more, such as four) pilot holes H in the bones B of the patient using a single drill device 14. In an initial state, the support plate 22 may be biased away from the guide plate 20, and the points 104 of the drill bits 30 may be arranged in the channels of the receivers 68 and/or in the guide holes 60. The points 104 may not extend beyond the engagement surface 64. The user can grasp the handle 74 in one hand and move the drill plate assembly 16 to position the guide plate 20 against the target area. Generally, two guide holes 60 and two drill bits 30 are arranged over each bone portion B with the alignment indicator 62 arranged or aligned with the cut between the bone portions B.

The user may then use the other hand to move the drill device 14 and insert the drill member 112 into the receiving shaft 100 to engage the flute 124 on the drill member 112 with the structure 120 in the receiving cavity 110. The user can activate the drill device 14, which may activate a motor 132 to rotate the drill member 112. The rotation of the drill member 112 may be transferred to the receiving shaft 100, causing the receiving shaft 100 and the drive gear 26 to rotate with the drill member 112. The drive gear 26 may cause the simultaneous rotation of the follower gears 28 and, consequently, the drill bits 30 coupled with the follower gears 28. Accordingly, the drill device 14 may cause simultaneous rotation of the multiple drill bits 30 of the drill plate assembly 16 through the gear assembly 24.

The user can apply the force F to the support plate 22. In non-limiting examples, the user can press the drill member 112 into or against the receiving shaft 100. The engagement between the receiving shaft 100 and the support plate 22 may cause the receiving shaft 100 and the support plate 22 to move as a single component. Accordingly, the pressing force F applied to the receiving shaft 100 may cause the support plate 22 to be pressed or moved toward the guide plate 20. The support plate 22 may move along the support shafts 82, with the pressing force F overcoming the biasing force of the biasing members 32. The biasing force may assist in providing a more even transfer of force F across the plate assembly 16. In this way, the centrally applied pressing force F may be more evenly distributed across the support plate 22 and, therefore, more evenly to the drill bits 30. This may also result in smoother movement of the components of the drill plate assembly 16.

The pressing force F causing the support plate 22 to move toward the guide plate 20 may move the drill bits 30 in the insertion direction through the guide holes 60 and into the bones B. The movement into or toward the bones B and the concurrent rotation of the drill bits 30 may cause the drill bits 30 to form the pilot holes H. The more evenly distributed pressing force F may assist with more evenly and uniformly applying pressure to the drill bits 30 to more evenly form the pilot holes H, which may be advantageous for receiving the bridged staple 12 (FIG. 9) or the fixation members 58 (FIG. 15). The insertion movement of the drill bits 30 may be guided by the receivers 68.

The support plate 22 may continue to be moved toward the guide plate 20 until the pilot holes H are formed at a selected depth. The receivers 68 may act as stoppers such that the support plate 22 abutting the receivers 68 stops the movement of the support plate 22. In such examples, the depth of the pilot holes H corresponds to the distance the support plate 22 may move to abut the receivers 68. It is also contemplated that other features may define the movement of the support plate 22 and the depth of the pilot holes H, such as the stopper 130 on the drill member 112, stoppers on the drill bits 30, or other features between the support plate 22 and the guide plate 20.

As the user releases or decreases the pressing force F, the biasing force may be configured to bias the support plate 22 away from the guide plate 20 (in a direction opposite the force F). In this way, the applied force F from the user may overcome the biasing force and as the user begins to remove or decrease the applied force F, the biasing force may move the support plate 22 toward the initial state abutting the caps 88. As the support plate 22 moves away from the guide plate 20, the support plate 22 may be configured to draw the drill bits 30 in the removal direction (opposite the insertion direction and the force F) out of and/or away from the bones B to disengage the drill plate assembly 16 from the target area. The drill plate assembly 16, driven by the drill device 14, forms multiple pilot holes H simultaneously, which may provide more consistent spacing and arrangement of the pilot holes H for the bridged staple 12 (FIG. 9) and decrease the time taken during the surgical procedure to form the pilot holes H.

Referring to FIGS. 9-12, the multiple pilot holes H may be formed for the insertion or implantation of the bridged staple 12 into the bones B to provide the consistent compressive force F between the bones B for a healing/recovery process. In the illustrated configuration, the bridged staple 12 includes the first and second prongs 40a, 40b coupled via the first connector 42, forming the first staple portion 48. Additionally, the bridged staple 12 may also include the third and fourth prongs 40c, 40d coupled via the second connector 44, forming the second staple portion 50. Each of the staple portions 48, 50 may be configured to be inserted into the bones B on opposing sides of the cut to provide the inward compressive force.

The bridged staple 12 may also include at least one bridge 46 for coupling the staple portions 48, 50 together. In this regard, the bridged staple 12 may be a multi-pronged staple with the prongs 40 connected by one or more bridges 46. In the illustrated configuration, the bridged staple 12 includes four prongs 40a-40d and two bridges 46, but it is contemplated that fewer or additional prongs 40 and/or fewer or additional bridges 46 may be utilized without departing the teachings herein.

Referring to FIGS. 9-11, the illustrated configuration of the bridged staple 12 includes two bridges 46. The bridges 46 may extend between and couple the two connectors 42, 44, which couples the two staple portions 48, 50 together while allowing movement of the prongs 40. The bridges 46 may be spaced from one another and may extend generally parallel to one another. This configuration forms a central opening 150 between the two bridges 46 and the two connectors 42, 44. This opening 150 may reduce the material that is implanted into the patient and may increase the elastic deformation properties of the bridged staple 12. It is contemplated that a single bridge 46 or multiple bridges 46 may be utilized without departing from the teachings herein.

The bridges 46 may be coupled to the first and second connectors 44 proximate to rounded corners 152, 154 of the staple portions 48, 50. The configuration with two bridges 46 may provide more rigidity to the staple 12 and less twisting may occur between the two staple portions 48, 50. Further, the bridges 46 may reduce translational forces between the two staple portions 48, 50 and may provide the function of a rigid, plate-like component without additional hardware being inserted into the patient.

The first prong 40a and the second prong 40b are generally coupled with or transition to the first connector 42 by the rounded corners 152. Similarly, the third prong 40c and the fourth prong 40d may be coupled to or transition to the second connector 44 via the rounded corners 154. The rounded corners 152, 154 may extend outside of the bridges 46. Accordingly, the staple portions 48, 50 may be wider than the spacing of the bridges 46 for engagement with the inserter 18 (FIG. 12). The rounded corners 152, 154 may reduce the profile of the staple 12 inserted into the patient, reducing sharp edges or locations that may protrude.

Each of prongs 40 may include a stepped engagement feature 158 along an inner surface thereof. The engagement features 158 may be advantageous for gripping the bone B to increase engagement between the staple 12 and the bone B once the staple 12 is inserted into the pilot holes H. The stepped engagement features 158 may assist with reducing the movement of the bridged staple 12 after insertion. Each of the prongs 40 may also include a pointed tip 160, which may assist in inserting the bridged staple 12 into the pilot holes H.

The bridged staple 12 may be constructed of nitinol, polyether ether ketone (PEEK), or other similar material. The construction of the bridged staple 12 may allow the prongs 40 to be elastically deformed. The bridged staple 12 may have sufficient elasticity to conform to the anatomy with sufficient rigidity for fixation and compression of the bone B. Accordingly, the bridged staple 12 may be biased to a first position or state, can be adjusted to a second position or state in response to an applied force, and can automatically return to the first state in response to the biasing force formed by the construction/material of the bridged staple 12. For example, the first and second prongs 40a, 40b may be biased toward the first or state where the prongs 40a, 40b are angled toward one another. This may be a closed or compressive state. In response to an applied force, the prongs 40a, 40b may be moved away from one another to be generally parallel with one another. The prongs 40a, 40b, the corners 152, and/or the first connector 42 may be configured to bend or otherwise adjust to move the prongs 40a, 40b to this widened insertion state.

In response to the applied force being removed from the prongs 40a, 40b, the prongs 40a, 40b may be configured to automatically return to the narrower, initial state. The third and fourth prongs 40c, 40d may be configured similarly. The adjustment of the prongs 40 between the states may be utilized for inserting the bridged staple 12 and then providing the compressive force on the bones B.

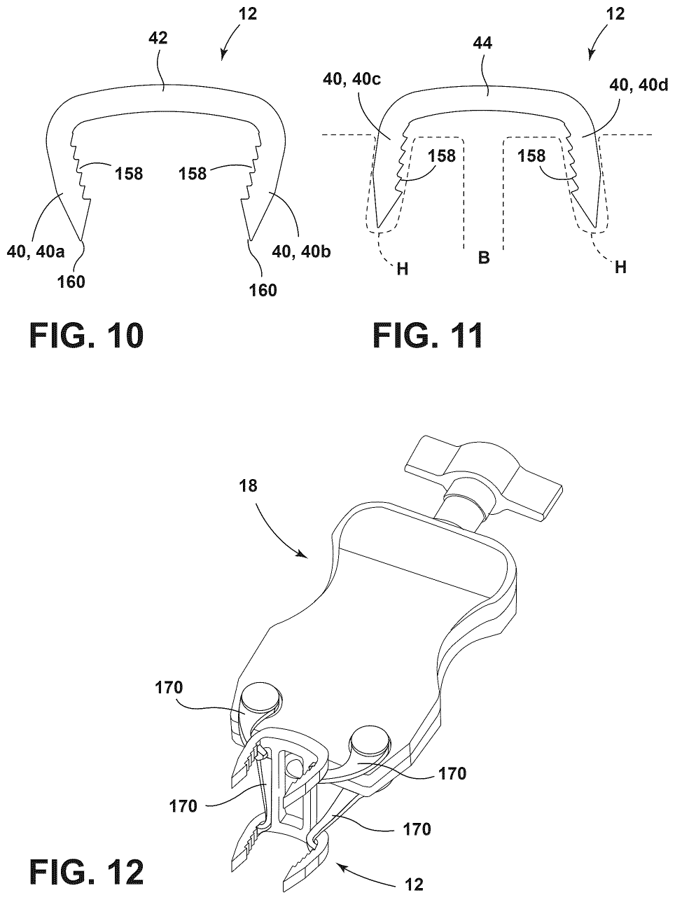

Referring now to FIGS. 10-12, in various aspects, the staple 12 may be inserted utilizing the inserter 18. The inserter 18 may include hooks 170 that engage the staple 12 proximate to the rounded corners 152, 154. Accordingly, the inserter 18 may include four hooks 170 for moving the four prongs 40. The hooks 170 may be configured to engage the prongs 40 from proximate to the bridges 46. In such examples, two hooks 170 face a first direction to engage the first staple portion 48, and two hooks 170 face a second direction to engage the second staple portion 50. The inserter 18 may be configured to engage and adjust the four prongs 40 simultaneously. In certain aspects, the inserter 18 may be configured to engage the staple 12 proximate to the corners 152, 154 outside of the bridges 46 or from between the bridges 46.

The inserter 18 can be activated or adjusted by the user to apply the force to the inner surface of the prongs 40, adjusting the prongs 40 outward to the parallel configuration. The parallel prongs 40 may then be inserted into the pilot holes H using the inserter 18. The first and third prongs 40a, 40c may be inserted into the pilot holes H in the first bone B on the first side of the cut, and the second and fourth prongs 40b, 40d may be inserted in the pilot holes H in the second bone B on the second side of the cut. In this way, bridged staple 12 may engage two bones B with the first and second connectors 42, 44 extending over the cut. The bridges 46 may extend generally perpendicular to the connectors 42, 44 and generally parallel to the cut.

Once the prongs 40 of the staple 12 are positioned in the pilot holes H, the inserter 18 may be disengaged from the staple 12. Disengaging the inserter 18 may remove the force F applied to the prongs 40, allowing the prongs 40 to return to or toward the original, angled state, which can provide the compressive inward force on each bone B or on each side of the cut of the bone B. The bridged staple 12 can provide constant compression from the prongs 40, while reducing transitional forces due to the connectors 42, 44 being interconnected via the bridges 46.

Referring to FIGS. 1-12, the surgical kit 10 may be used for one or more medical procedures that utilize pilot holes H and multi-pronged staples 12 inserted into bones B or bone portions B. The drill plate assembly 16 may be utilized with the single drill device 14 for simultaneously drilling multiple pilot holes H in bone B. This may be advantageous for decreasing the time for the surgical procedure and increasing accuracy in the positioning of the pilot holes H. Further, this may reduce components needed for the surgical procedure by omitting more drill guides. The inserter 18 may be utilized to adjust and insert the bridged staple 12 into the pilot holes H. The bridged staple 12 may be configured to provide consistent, inward, compressive force to the bones B on either side of the cut or break. Further, the bridges 46 may provide more rigidity to the bridged staple 12 for the procedure and may reduce translational forces that may affect more two-legged staples, as well as utilize fewer implanted components.

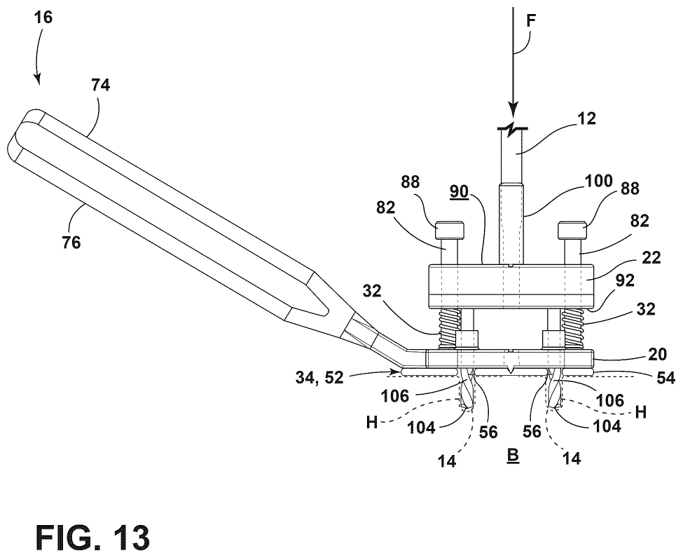

Referring to FIG. 13, the drill plate assembly 16 can be to drill the pilot holes H for the sternal plate 52. The drill plate assembly 16 can be positioned against the bone B and form the pilot holes H as described herein. Additionally or alternatively, the sternal plate 52 can be positioned on the bone B or bone B portions. The sternal plate 52 can be positioned at the location where the plate 52 is to be implanted. The drill plate assembly 16 can be positioned over or on the sternal plate 52. The openings 60 and the drill bits 30 are aligned with the apertures 56 of the sternal plate 52. The indicator 62 may assist with aligning the sternal plate 52. The receivers 68 may extend below the guide plate 20 to be positioned in the apertures 56. This may align the apertures 56 with the drill bits 30 and may also reduce movement between the drill plate assembly 16 and the sternal plate 52.

The force F may be applied to the support plate 22, moving the support plate 22 toward the guide plate 20. The drill bits 30 may be moved in the insertion direction, through the guide holes 60, through the apertures 56, and into the bone B to form the pilot holes H. Upon release of the force F, the biasing force of the biasing members 32 may adjust the support plate 22 to the initial position away from the guide plate 20. The drill plate assembly 16 can be removed from the sternal plate 52 and the surgical site. The fixation members 58 may then be inserted through the apertures 56 and into the bone B to attach the sternal plate 52 to the bone B. An additional insertion device may be included in the kit 10 for inserting or implanting the fixation members 58.

The drill plate assembly 16 may be separate from the sternal plate 52. It is also contemplated that the sternal plate 52 may be pre-loaded onto the drill plate assembly 16, which may assist with properly aligning the apertures 56 with the drill bits 30. Further, in certain aspects, the fixation members 58 may also be pre-loaded onto the sternal plate 52. The pre-loaded configurations may maximize efficiency during the procedure and assist with alignment.

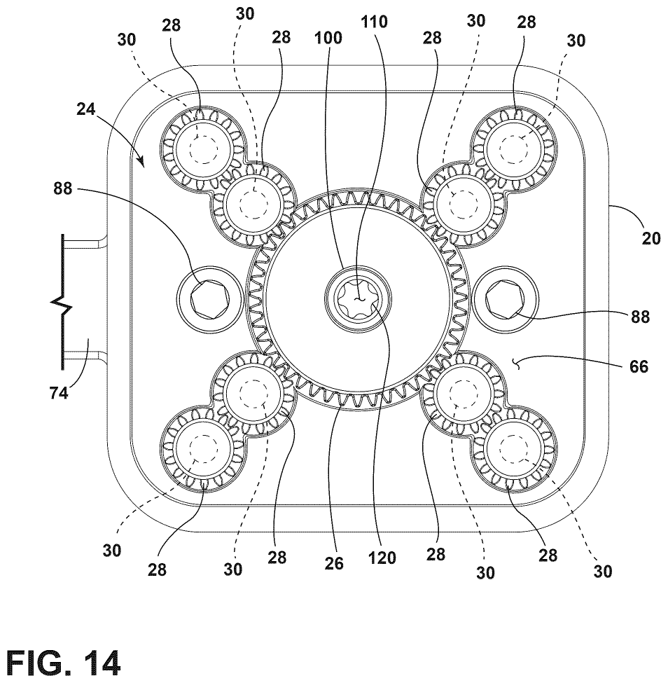

Referring to FIG. 14, the configuration of the drill plate assembly 16 may generally correspond to the pattern of the apertures 56 of the sternal plate 52. For example, at least one of the follower gears 28 may operate as a drive gear for another follower gear 28. In such configurations, the drive gear 26 may cause rotation of the first follower gear 28, which can, consequently, cause rotation of an adjacent follower gear 28 that is spaced from the drive gear 26. This may allow more follower gears 28 and/or different arrangements of follower gears 28 to simultaneously drill the pilot holes H for the sternal plate 52.

Referring to FIG. 15, the surgical kit 10 may further include, or be used in conjunction with, a second closure assembly 180 or compression assembly. The second closure assembly 180 may be a compression wrap, a suture, or suture tape (e.g., Fibertape®). The second closure assembly 180 may be used in conjunction with the bridged staples 12 (e.g., overlapping, alternating, etc.) and/or the sternal plate 52 to compress the tissue about a perimeter of the corresponding anatomy. For example, the second closure assembly 180 may assist in reducing a gap or space between two bone portions B. The second closure assembly 180 may also provide a different type of fixation compared to the bridged staple 12 or sternal plate 52. In such examples, the second closure assembly 180 may provide a circumferential fixation and the bridged staples 12 or sternal plate 52 may provide a consistent compression against lateral forces.

The second closure assembly 180 may include one or more closure bands that may wrap around a perimeter of the tissue through which the incision is made and bind the sectioned sides of the anatomy (e.g., sternal sections). In this way, one or more bands of the second closure assembly 180 may compress the sectioned sides to retain the anatomy to receive the staples 12. The second closure assembly 180 may also extend through pilot holes H in the bone portions B, such as those formed by the drill plate assembly 16.

In certain aspects, the second closure assembly 180 may be applied prior to the implant 34 and in other aspects, the implant 34 may be applied prior to the second closure assembly 180. The implant 34 and the second closure assembly may overlap. In such examples, the bridged staple(s) 12 or the sternal plate 52 may be inserted over the second closure assembly 180. Alternatively, the second closure assembly 180 may be applied over the inserted bridged staple(s) 12 or sternal plate 52. In further non-limiting examples, the implant 34 and the second closure assembly 180 may be disposed adjacent to one another. In such examples, the implant 34 and the second closure assembly 180 may not directly overlap. The second closure assembly 180 and the implant(s) 34 may form an alternating pattern over the incision or space between the bone portions B to provide alternating types of compression along the bone portions B. For example, the second closure assembly 180 may extend across the incision at two locations and the bridged staple 12 and/or sternal plate 52 may be disposed between the two locations. The use of two or more compressive features may be advantageous to provide different types of compression to fix the bone portions B together.

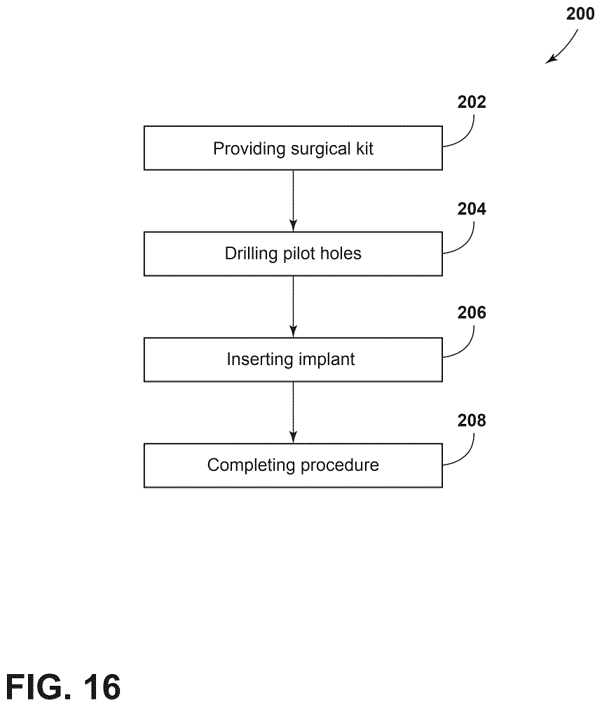

Referring to FIG. 16, as well as FIGS. 1-15, each of the components of the surgical kit 10 may be utilized for a method 200 of fixing bones B or bone portions B together. The overall method 200 of fixing the bones B may include providing the surgical kit 10 (202). The surgical kit 10 may include the drill device 14 with the drill member 112, the drill plate assembly 16, the bridged staple(s) 12, the inserter 18, the sternal plate 52, the fixation members 58, and/or the insertion device for the fixation members 58. The method 200 may also include drilling the pilot holes H (204) and inserting the implant 34 (206). For example, inserting the implant 34 can include inserting the bridged staples 12. Inserting the implant 34 can also include positioning the sternal plate 52 on the bone B and fixing the sternal plate 52 to the bone B with the fixation member 48. Each of these steps (204, 206) can include multiple steps or sub-steps of the overall method 200. In certain aspects, the method 200 may also include applying the second closure assembly 180. The method 200 may end with completing the surgical procedures (208). The method 200 may provide for the internal fixation of the bones B with a consistent compressive force by the bridged staple 12, the sternal plate 52, and/or the second closure assembly 180.

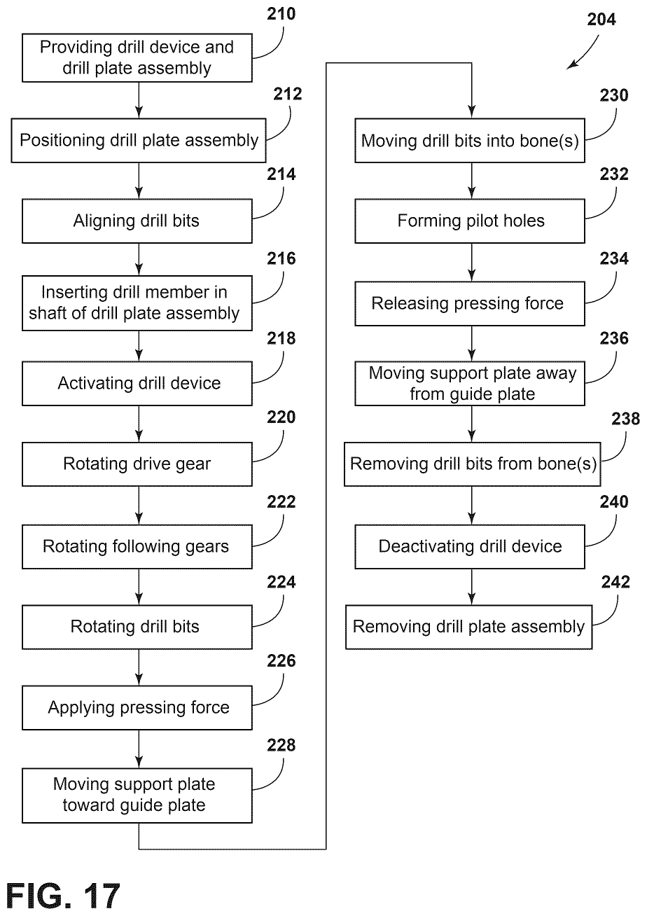

Referring to FIG. 17, as well as FIGS. 1-16, the step 202 of drilling the pilot holes H may also be referred to as a method 202 of drilling the pilot holes H. The drill plate assembly 16 and the drill device 14 may be provided (210). The drill member 112 may be selected based on the shape/configuration of the end to mate or interlock with the shaft 100 of the drill plate assembly 16. The user may grasp the handle 74 of the drill plate assembly 16 and position the engagement surface 64 of the plate assembly 16 against the target area (212). The target area may generally correspond to tissue or bones B with an incision, cut, or break that is to be closed or fixed together. The target area may also include the sternal plate 52 positioned over the bone B. In certain aspects, two guide holes 60 and two drill bits 30 may be arranged to engage the first bone, and the remaining two guide holes 60 and two drill bits 30 may be arranged to engage the second bone (214). The alignment indicator 62 may be arranged over the space or cut between the bones B.

The user may position the end of the drill member 112 within the receiving cavity 110 of the shaft 100 (216). The user may rotate the drill device 14 and/or drill member 112 to align the drill member 112 and interlock the drill member 112 with the structure(s) 120 in the receiving shaft 100. It is contemplated that the user may hold the drill plate assembly 16 in one hand and the drill device 14 in the other hand. Alternatively, one medical professional may hold the drill device 14 while another medical professional may hold the drill plate assembly 16.

The drill device 14 may be activated (218), activating the motor 132 to rotate the drill member 112. The rotation of the drill member 112 may cause or drive rotation of the drive gear 26 based on the engagement between the drill member 112 and the shaft 100 (220). In other words, the rotational force may be transferred from the drill member 112 to the drive gear 26. The rotation of the drive gear 26 may cause rotation of the follower gears 28 based on the engagement between the drive gear 26 and the follower gears 28 (222). Generally, the drive gear 26 may simultaneously and equally engage each of the follower gears 28 to generate simultaneous and equal rotation of the follower gears 28. With the drill bits 30 coupled to the follower gears 28, the rotation of the follower gears 28 causes or drives rotation of the drill bits 30 (224). Accordingly, the rotational force of the drill member 112 is transferred through the gear assembly 24 to the drill bits 30. The configuration of the gear assembly 24 may allow the drill bits 30 to rotate at the same speed.

The user may apply the pressing force F to the support plate 22, overcoming the biasing force of the biasing members 32 (226). The pressing force F may be described as a downward or closing force F where the user can press on the drill device 14 to transfer the pressing force F to the receiving shaft 100 and, consequently, the support plate 22. The pressing force F may cause the support plate 22 to move along the support shafts 82 and toward the guide plate 20 (228). The engagement between the receiving cavity 110 and the drill member 112 may transfer the pressing force F to the support plate 22. The pressure may be applied at a central location on the support plate 22 and may be evenly distributed due to the biasing force of the biasing members 32 at the outer ends of the support plate 22. The support plate 22 may be configured to move closer to the guide plate 20 with movement being guided or stabilized by the support shafts 82 extending through the support plate 22.

In response to the movement of the support plate 22, the drill bits 30 may be configured to move through the receivers 68 and the guide holes 60 and into the bone B (230). The simultaneous or concurrent pressing force F moving the drill bits 30 into the bone and rotation of the drill bits 30 may cause the formation or drilling of the pilot holes H in the two bones B (232). This may result in two pilot holes H in each bone B being formed simultaneously using a consistent speed of the drill bits 30 and consistent pressure applied to the drill bits 30. This may be advantageous for simultaneously forming or drilling multiple substantially identical pilot holes H. Moreover, the receivers 68 may assist in retaining the drill bits 30 perpendicular to the guide plate 20, for forming straight/vertical pilot holes H into the bone (rather than angled pilot holes H).

The user may release or reduce the pressing force F applied to the plate assembly 16 (234). In response to the reduction in force F, the biasing force of the biasing members 32 may be configured to move the support plate 22 along the support shafts 82 and away from guide plate 20 (236). The support plate 22 may continue to move until the outer surface 90 of the support plate 22 abuts the caps 88, limiting the movement caused by the biasing member 32. The movement of the support plate 22 may move or draw the drill bits 30 away from the bones B, removing the drill bits 30 from the target area (238). As the biasing members 32 returns the support plate 22 to the initial state abutting the caps 88, the drill bits 30 may be fully withdrawn from the target area. The drill device 14 may be deactivated (240), and the drill plate assembly 16 may be removed from the target area (242). This method 204 may simultaneously drill multiple pilot holes H into the bones B at the target area, which can be drilled in an arrangement that matches the configuration of the bridged staple 12.

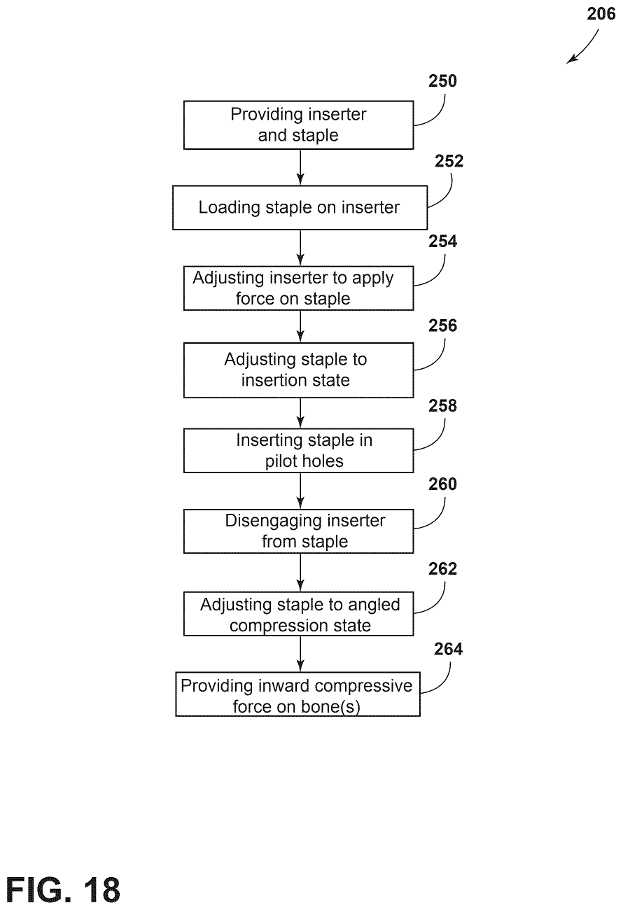

Referring to FIG. 18, as well as FIGS. 1-17, the step 206 of inserting the implant 34 can include inserting the bridged staple 12, which may also be referred to as a method 206 of inserting the staple 12. The inserter 18 and the bridged staple 12 may be provided (250). The bridged staple 12 may include the four prongs 40. The four prongs 40 may generally align with the arrangement of the pilot holes H formed by the drill plate assembly 16. The first staple portion 48 may include two prongs 40a, 40b coupled via the first connector 42, and the second staple portion 50 may include two prongs 40c, 40d coupled via the second connector 44. The first and second staple portions 48, 50 may be coupled via the bridge(s) 46.

The staple 12 may be loaded on the inserter 18 (252). The bridged staple 12 may be in the first state, with the staple portions 48, 50 in the inwardly angled state. The hooks 170 of the inserter 18 may be arranged proximate to the respective corners 152, 154 to engage the prongs 40 of the bridged staple 12. The hooks 170 may be configured to engage each of the four prongs 40 at a single time. The inserter 18 may be activated or adjusted to move the hooks 170 outward against the prongs 40 concurrently (254). The inserter 18 may be configured to move the prongs 40 outward to the second/insertion state with the four prongs 40 in the generally parallel configuration (256).

The prongs 40 of the bridged staple 12 may be inserted into the pilot holes H in the bones B (258). Typically, the bridged staple 12 may be inserted with the inserter 18 or otherwise retained in the parallel configuration or insertion state. The pointed tips 160 may assist with the insertion, and the engagement features 158 may engage the bones B for retaining the prongs 40 in the pilot holes H. The bridged staple 12 may be pressed, stamped, or otherwise moved into the pilot holes H and the bones B until the connectors 42, 44 abut an outer surface of the bones B. Each staple portion 48, 50 may engage each bone B. Accordingly, the first and third prongs 40a, 40c may be inserted into the first bone B, and the second and fourth prongs 40b, 40d may be inserted into the second bone B. The connectors 42, 44 may extend over the cut or space between the bones B.

The inserter 18 may be disengaged from the inserted bridged staple 12 (260). Upon release of the outward force by the inserter 18, the prongs 40 may be configured to adjust to or toward the inwardly angled position due to the biased configuration or biasing force of the bridged staple 12 (262). The first and second prongs 40a, 40b may be configured to move toward one another, and the third and fourth prongs 40c, 40d may be configured to move toward one another. This movement may provide the inward compressive force F on the bones B toward one another (264). The bridged staple 12 may, at least temporarily, remain in the patient to provide internal fixation of the bones B. In certain aspects, the second closure assembly 180 may also be used, which may also, at least temporarily, remain in the patient to provide internal fixation with a second type of compression.

The step 206 of inserting the implant 34 can include inserting the sternal plate 52, which may also be referred to as a method 206 of inserting the staple 12. The sternal plate 52 may be positioned to align the apertures 56 with the pilot holes H. The fixation members 58 may be positioned through the apertures 56 and inserted into the pilot holes H. The insertion device may be utilized to insert the fixation members 58 into the bone B. In certain aspects, the second closure assembly 180 may also be used, which may also, at least temporarily, remain in the patient to provide internal fixation with a second type of compression.

The method 204 of drilling the pilot holes H and the method 206 of inserting the implant 34 may be repeated based on the number of implants 34 used during the surgical procedure. The surgical process may then be completed (208). The methods 200, 204, 206 may be modified or conducted in any order, with steps omitted, repeated, performed concurrently, etc. without departing from the teachings herein.

Referring to FIGS. 1-18, the drill plate assembly 16 may be utilized for simultaneously drilling multiple pilot holes H into multiple bones B using a single drill device 14. The drill plate assembly 16 may also include the biasing member 32 for simultaneously removing the drill bits 30 from the target area. The drill bits 30 may be arranged in a predefined configuration or pattern to match or correspond with the position/arrangement of the prongs 40 in the parallel configuration (e.g., in the widened insertion state) or the apertures 56 of the sternal plate 52. For procedure using the bridged staple 12, the prongs 40 of the bridged staple 12 may be inserted into the pilot holes H simultaneously. The bridges 46 may interconnect the staple portions 48, 50, reducing transnational forces and providing increased rigidity to the implanted staple 12 without additional hardware.

Use of the present device may provide for a variety of advantages. For example, the multi-pronged staple 12 may reduce hardware implanted in the patient during the surgical procedure. Further, the bridged connection of the staple portions 48, 50 may function similarly to a rigid plate. The bridges 46 may also retain the relationship between the two staple portions 48, 50, which may be advantageous for internally fixing the bones B and retaining the relationship between the bones B. The sternal plate 52 can promote faster healing and increased compressive force with the fixation members 58.

Further, the drill plate assembly 16 may be utilized to simultaneously or concurrently drill multiple pilot holes H in a predefined pattern with a single drill device 14. This may be more efficient for the surgical team during the surgical process compared to other processes of sequentially drilling individual pilot holes H. Further, the simultaneous drilling may also assist with better alignment of the pilot holes H rather than of a simple drill or alignment guides which can inadvertently shift between the drilling of the individual pilot holes H. Moreover, the receivers 68 may act as stoppers for the movement of the support plate 22 and, consequently, the drill bits 30 to define a depth of the pilot holes H. Additionally, the biasing members 32 may assist with withdrawing the drill bits 30 from the target area. The biasing members 32 may also assist in distributing the pressure applied by the user to more evenly apply pressure for drilling the pilot holes H. Additional benefits or advantages may be realized and/or achieved.

The devices and methods disclosed herein are further summarized in the following paragraphs and are further characterized by combinations of any and all various aspects described herein.

According to an aspect of the present disclosure, a surgical kit may include a drill plate assembly configured to drill multiple pilot holes into bones. The drill plate assembly may include a guide plate configured to be positioned against a target area. The guide plate may define guide holes. A support plate may define an aperture. A gear assembly may include a receiving shaft extending through the aperture. A drive gear may be coupled with the receiving shaft, and follower gears may be engaged with the drive gear. Each drill bit may be coupled with a respective one of the follower gears. Rotation of the drive gear may be configured to cause rotation of the follower gears and, consequently, rotation of the drill bits. The support plate may be configured to move toward the guide plate in response to an applied force to move the drill bits through the guide holes to engage the target area and drill the multiple pilot holes. The surgical kit may include an implant configured to extend into the multiple pilot holes.

According to another aspect of the present disclosure, an implant may include a bridged staple with a first pair of prongs coupled via a first connector and a second pair of prongs coupled via a second connector. At least one bridge may couple the first and second connectors. The first pair of prongs and the second pair of prongs may be configured to be inserted into the multiple pilot holes, respectively, to provide an inward compressive force between the bones.

According to another aspect of the present disclosure, an implant may include a sternal plate defining apertures and fixation members configured to extend through the apertures and into pilot holes.

According to an aspect of the present disclosure, an inserter may be configured to adjust a first pair of prongs and a second pair of prongs to a widened insertion states for insertion into multiple pilot holes.

According to an aspect of the present disclosure, each of a first pair of prongs and a second pair of prongs may be biased toward an inwardly angled state.

According to an aspect of the present disclosure, guide holes may be arranged proximate corners of a guide plate.

According to an aspect of the present disclosure, receivers may extend from an inner surface of a guide plate and toward a support plate.

According to an aspect of the present disclosure, a support plate may be configured to abut receivers in response to an applied force.

According to an aspect of the present disclosure, support shafts may be coupled to a guide plate and extend through holes defined in a support plate.

According to an aspect of the present disclosure, biasing members may be arranged about support shafts and configured to bias a support plate away from a guide plate.

According to an aspect of the present disclosure, a drill plate assembly may be configured to be engaged by a drill device, and the drill plate assembly may include a guide plate configured to be positioned against a target area. The guide plate may define guide holes. A handle may be coupled to the guide plate. A support plate may be spaced from the guide plate. A receiving shaft may extend through the support plate and may be configured to engage with said drill device. A drive gear may be operably coupled to the support plate. The drive gear may be operably coupled to the receiving shaft to be rotated by said drill device. Follower gears may be operably coupled to the drive gear. Drill bits may be operably coupled with the follower gears, respectively. Rotation of the drive gear may be configured to cause rotation of the follower gears and, consequently, rotation of the drill bits, and the drill bits may be configured to be moved through the guide holes to engage the target area in response to a force applied to the support plate to drill multiple pilot holes. At least one biasing member may extend between the guide plate and the support plate to bias the support plate away from the guide plate.

According to an aspect of the present disclosure, a handle may extend at an obtuse angle from a guide plate.

According to an aspect of the present disclosure, drill bits may be configured to be rotated simultaneously to simultaneously form multiple pilot holes.

According to an aspect of the present disclosure, at least one support shaft may extend between a guide plate and a support plate.

According to an aspect of the present disclosure, at least one support shaft may extend through a support plate.

According to an aspect of the present disclosure, a cap may be coupled to at least one support shaft proximate to an outer surface of a support plate. An outer surface may be configured to abut the cap in response to a biasing force of at least one biasing member.

According to an aspect of the present disclosure, at least one biasing member may be a coil spring arranged around at least one support shaft.

According to an aspect of the present disclosure, at least one biasing member may include first and second biasing members configured to engage opposing ends of a support plate.

According to an aspect of the present disclosure, a receiving shaft may define a receiving cavity with an interior surface defining structures configured to interlock with an end of a drill member of a drill device.

According to an aspect of the present disclosure, follower gears and drill bits may be arranged at corners of a support plate. A drive gear may be centrally located between the follower gears.

According to an aspect of the present disclosure, a guide plate may include an alignment indicator extending from an engagement surface.

According to an aspect of the present disclosure, a bridged staple may include a first staple portion including first and second prongs. Each of the first and second prongs may include a stepped engagement feature defined on an inner surface thereof. A first connector may couple the first and second prongs. The first and second prongs may be configured to be adjusted away from one another to an insertion state. The first and second prongs may be biased toward a compressive stage angled toward one another. A second staple portion may include third and fourth prongs. Each of the third and fourth prongs may include a stepped engagement feature defined on an inner surface thereof. A second connector may couple the third and fourth prongs. The third and fourth prongs may be configured to be adjusted to an insertion state. The third and fourth prongs may be biased toward a compressive state angled toward one another. A first bridge may extend between the first and second connectors proximate to the first and third prongs. A second may extend between the first and second connectors proximate to the second and fourth prongs. The first and second staple portions may be wider than the first and second bridges.

According to an aspect of the present disclosure, each of first, second, third, and fourth prongs may define a pointed tip.

According to an aspect of the present disclosure, each of first and second prongs may be coupled with a first connector via rounded corners. Each of third and fourth prongs may be coupled with a second connector via rounded corners.

According to an aspect of the present disclosure, first and second bridges may be spaced from one another defining a central opening between the first and second bridges and first and second connectors.

According to an aspect of the present disclosure, first and second bridges may extend parallel to one another.

According to an aspect of the present disclosure, a method of drilling pilot holes may include: positioning a drill plate assembly against a target area; aligning a first pair of guide holes of a guide plate of the drill plate assembly on a first side of a cut between first and second bones; aligning a second pair of guide holes of the guide plate on a second side of the cut; engaging a drill device with a drive gear operably coupled with a support plate of the drill plate assembly; driving rotation of the drive gear with the drill device; driving follower gears with the drive gear; driving rotation of drill bits with the rotation of the follower gears; moving the drill bits through first and second pairs of guide holes, respectively, in response to a force applied to the support plate; and concurrently drilling the multiple pilot holes in the target area with the drill bits.

According to an aspect of the present disclosure, a method may include moving a support plate toward a guide plate.

According to an aspect of the present disclosure, a step of moving a support plate toward a guide plate may include overcoming a biasing force of a biasing member between the support plate and the guide plate.

According to an aspect of the present disclosure, a method may include moving a support plate away from a guide plate and drawing drill bits away from a target area in response to movement of the support plate.

According to an aspect of the present disclosure, a step of engaging a drill device with a drive gear may include inserting an end of a drill member of the drill device in a receiving cavity of a receiving shaft operably coupled with the drive gear and interlocking the end of the drill member with a structure on an interior surface of the receiving cavity.

According to an aspect of the present disclosure, a step of moving a support plate away from a guide plate may include moving the support plate with a biasing force of a biasing member between the support plate and the guide plate.

According to an aspect of the present disclosure, a method of drilling pilot holes may include: positioning first and second guide holes of a guide plate of a drill plate assembly on a first side of a cut between bone portions; positioning second and third guide holes of the guide plate on a second side of the cut; driving rotation of a drive gear with a drill device; driving rotation of follower gears with the rotation of the drive gear; driving rotation of drill bits with the rotation of the follower gears; moving the drill bits in a first direction through first, second, third, and fourth guide holes, respectively, in response to a force applied to a support plate of the drill plate assembly; drilling multiple pilot holes in a target area with the drill bits; and moving the drill bits in a second opposing direction away from the target area in response to a biasing force of a biasing member between the guide plate and the support plate.

According to an aspect of the present disclosure, a method may include applying a force to a support plate with a drill device.

According to an aspect of the present disclosure, a step of drilling multiple pilot holes may include drilling the multiple pilot holes simultaneously.

According to an aspect of the present disclosure, a method of fixing bone portions together may include: aligning guide holes of a drill plate assembly; driving rotation of a gear assembly of the drill plate assembly with a drill device; driving rotation of drill bits with the rotation of the gear assembly; moving the drill bits in an insertion direction through the guide holes, respectively, in response to a force applied to a support plate of the drill plate assembly; drilling a pair of pilot holes in each of first and second bone portions simultaneously with the drill bits; adjusting first and second pairs of prongs of a bridged staple outward to an insertion state; inserting the first pair of the prongs into the pair of pilot holes in the first bone portion and the second pair of prongs in the pair of pilot holes in the second bone portion; and applying an inward compressive force between the first bone portion and the second bone portion with the bridged staple.

According to an aspect of the present disclosure, a method may include applying a force to a support plate with a drill device and moving the support plate toward a guide plate of a drill plate assembly in response to the force applied to the support plate.

According to an aspect of the present disclosure, a method may include moving the support plate away from the guide plate with a biasing force of a biasing member and drawing the drill bits away from the target area in response to movement of the support plate away from the guide plate.

According to an aspect of the present disclosure, a step of drilling multiple pilot holes may include drilling the multiple pilot holes simultaneously.

According to an aspect of the present disclosure, a method of inserting a multi-prong staple into pilot holes in bones may include: simultaneously adjusting first and second pairs of prongs of a bridged staple outward to an insertion state; inserting the first pair of the prongs into a pair of pilot holes in a first bone portion and the second pair of prongs in a pair of pilot holes in a second bone portion; and applying an inward compressive force between the first bone portion and the second bone portion with the bridged staple.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents

1. A surgical kit, comprising:
a drill plate assembly configured to drill multiple pilot holes into bones, wherein the drill plate assembly includes:
a guide plate configured to be positioned against a target area, wherein the guide plate defines guide holes;
a support plate defining an aperture;
a gear assembly including a receiving shaft extending through the aperture, a drive gear coupled with the receiving shaft, and follower gears engaged with the drive gear; and
drill bits, wherein each drill bit is coupled with a respective one of the follower gears, and wherein rotation of the drive gear is configured to cause rotation of the follower gears and, consequently, rotation of the drill bits, and wherein the support plate is configured to move toward the guide plate in response to an applied force to move the drill bits through the guide holes to engage the target area and drill the multiple pilot holes; and
an implant configured to be inserted into the multiple pilot holes.

2. The surgical kit of claim 1, wherein the implant includes:
a bridged staple including:
a first pair of prongs coupled via a first connector;
a second pair of prongs coupled via a second connector; and
at least one bridge coupling the first and second connectors,
wherein the first pair of prongs and the second pair of prongs are configured to be inserted into the multiple pilot holes, respectively, to provide an inward compressive force between the bones.

3. The surgical kit of claim 1, wherein the implant includes:
a sternal plate defining apertures; and
fixation members configured to extend through the apertures and into the pilot holes, respectively.

4. The surgical kit of claim 1, wherein the guide holes are arranged proximate corners of the guide plate.

5. The surgical kit of claim 1, further comprising:
receivers extending from an inner surface of the guide plate and toward the support plate.

6. The surgical kit of claim 5, wherein the support plate is configured to abut the receivers in response to the applied force.

7. The surgical kit of claim 1, further comprising:
support shafts coupled to the guide plate and extending through holes defined in the support plate.

8. The surgical kit of claim 7, further comprising:
biasing members arranged about the support shafts and configured to bias the support plate away from the guide plate.

9. A drill plate assembly configured to be engaged by a drill device, the drill plate assembly comprising:
a guide plate configured to be positioned against a target area, wherein the guide plate defines guide holes;
a handle coupled to the guide plate;
a support plate spaced from the guide plate;
a receiving shaft extending through the support plate and configured to engage with said drill device;
a drive gear operably coupled to the support plate, wherein the drive gear is operably coupled to the receiving shaft to be rotated by said drill device;
follower gears operably coupled to the drive gear;
drill bits operably coupled with the follower gears, respectively, wherein rotation of the drive gear is configured to cause rotation of the follower gears and, consequently, rotation of the drill bits, and wherein the drill bits are configured to be moved through the guide holes to engage the target area in response to a force applied to the support plate to drill multiple pilot holes; and
at least one biasing member extending between the guide plate and the support plate to bias the support plate away from the guide plate.

10. The drill plate assembly of claim 9, wherein the handle extends at an obtuse angle from the guide plate.

11. The drill plate assembly of claim 9, wherein the drill bits are configured to be rotated simultaneously to simultaneously form the multiple pilot holes.

12. The drill plate assembly of claim 9, further comprising:
at least one support shaft extending between the guide plate and the support plate.

13. The drill plate assembly of claim 12, wherein the at least one support shaft extends through the support plate.

14. The drill plate assembly of claim 13, further comprising:
a cap coupled to the at least one support shaft proximate to an outer surface of the support plate, and wherein the outer surface is configured to abut the cap in response to a biasing force of the at least one biasing member.

15. The drill plate assembly of claim 12, wherein the at least one biasing member is a coil spring arranged around the at least one support shaft.

16. The drill plate assembly of claim 9, wherein the at least one biasing member includes first and second biasing members configured to engage opposing ends of the support plate.

17. The drill plate assembly of claim 9, wherein the receiving shaft defines a receiving cavity with an interior surface defining structures configured to interlock with an end of a drill member of said drill device.

18. The drill plate assembly of claim 9, wherein the follower gears and the drill bits are arranged at corners of the support plate, and wherein the drive gear is centrally located between the follower gears.

19. The drill plate assembly of claim 9, wherein the guide plate includes an alignment indicator extending from an engagement surface.

20. A method of drilling pilot holes, comprising:
positioning a drill plate assembly against a target area;
aligning a first pair of guide holes of a guide plate of the drill plate assembly on a first side of a cut between first and second bone portions;
aligning a second pair of guide holes of the guide plate on a second side of the cut;
engaging a drill device with a drive gear operably coupled with a support plate of the drill plate assembly;
driving rotation of the drive gear with the drill device;
driving rotation of follower gears with the rotation of the drive gear;
driving rotation of drill bits with the rotation of the follower gears;
moving the drill bits through first and second pairs of guide holes, respectively, in response to a force applied to the support plate; and
concurrently drilling multiple pilot holes in the target area with the drill bits.
